(19) United States
(12) Patent Application Publication
(10) Pub. No.: US 2006/0280706 A1
(43) Pub. Date: Dec. 14, 2006

(54) Rinse-off cosmetic composition containing interference particles

(75) Inventor: Laurence Sebillotte-Arnaud, L'Hay Les Roses (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/440,131

(22) Filed: May 25, 2006

Related U.S. Application Data
(60) Provisional application No. 60/688,349, filed on Jun. 8, 2005.

(30) Foreign Application Priority Data
May 25, 2005 (FR) 05 51371

(51) Int. Cl. A61K 8/29; A61K 8/26

(52) U.S. Cl. 424/63; 424/401

(57) ABSTRACT

A rinse-off cosmetic composition in the form of an emulsion containing an oily phase and an aqueous phase, and containing hydrophilic interference particles chosen from uncolored nacres coated with one or more coats of one or more metal oxides. The composition according to the invention changes color during application to the skin and thus makes it possible to assess the efficacy of the removal of makeup or of the care of the skin.

### REFERENCE TO PRIOR APPLICATIONS

This application claims priority to U.S. provisional application 60/688,349 filed Jun. 8, 2005, and to French patent application 0551371 filed May 25, 2005, both incorporated herein by reference.

### FIELD OF THE INVENTION

The invention relates to a cosmetic composition containing interference particles as efficacy indicators, and to its use as a product for cleansing or for removing makeup from the skin of the face and/or the body, including the scalp, and the hair, or as a skincare product. Preferably, the invention compositions are rinse-off products.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing of the skin is very important for facial care, and should be as efficient as possible, since greasy residues such as excess sebum, the residues of the cosmetic products used daily and makeup products, especially waterproof products, accumulate in the folds of the skin and can block the skin pores and lead to the formation of spots.

To obtain good makeup removal from the skin, it is known practice to use makeup-removing emulsions in the form of milks or creams. However, it is not always easy to know what amount of product should be applied to the face or how long the massaging should last in order for the makeup removal to be optimal. It is important though for the skin to be completely freed of makeup for the reasons indicated above.

### OBJECTS OF THE INVENTION

There is thus still a need for a product which, while removing makeup or cleansing, indicates the efficacy of the makeup removal or of the cleansing, and which allows the user of makeup products to know whether or not the makeup removal is sufficient.

### SUMMARY OF THE INVENTION

It has now been discovered, surprisingly, that the objects of the invention can be achieved by adding hydrophilic interference particles to makeup-removing products. These pigments change color gradually as they are applied, thus indicating by this color change the efficacy of the makeup removal. Moreover, the addition of these pigments makes the makeup removal more entertaining. This color-change effect may also be used to indicate the efficacy of the rinsed-off care products.

Rinse-off cosmetic compositions containing shiny pigments have already been described, especially in documents U.S. Pat. No. 6,759,376 and WO-A-2004/100 921. However, these are generally pigments that have been made hydrophobic, and, what is more, the aim is to deposit these pigments onto the skin, the hydrophobic modification of the particles and the presence of emollient promoting the remanence of these pigments after they have been rinsed off, which leads to a shiny deposit on the skin. In addition, these pigments do not give a color-change effect during application to the skin.

In contrast, by virtue of their hydrophilic nature, the pigments used in the present invention do not leave a shiny deposit on the surface of the skin after rinsing, since this deposit would then be-synonymous with poorly cleansed skin or skin from which the makeup has been poorly removed in the context of the present invention.

### DETAILED DESCRIPTION OF THE PREFERRED EMBODIMENTS

One subject of the present patent application is thus a rinse-off cosmetic composition in the form of an emulsion containing an oily phase and an aqueous phase, wherein it contains hydrophilic interference particles chosen from uncolored nacres coated with one or more coats of one or more metal oxides, these nacres having a maximum length of less than or equal to 130 μm, the thickness of metal oxides being greater than 80 nm, the amount of inner phase being greater than 50% by weight relative to the total weight of the composition.

Since the composition is preferably intended for topical application, it comprises a physiologically acceptable medium. Moreover, the term “physiologically acceptable medium” means a medium that is compatible with the skin, the lips, the scalp, the eyelashes, the eyes, the nails and/or the hair.

The composition according to the invention has the advantage of being easy to apply, of allowing quick and efficient makeup removal, of being easy to remove without leaving the skin greasy or shiny and of leaving the skin comfortable after use (no tautness or dryness).

The compositions of the invention are preferably rinse-off compositions (rinsing with water or with a tonic), and they may be used in the field of removal of makeup from or cleansing of the skin of the face or the body, the hair, including the scalp, and mucous membranes such as the lips. They may also constitute care products, for example rinse-off masks (in the usual manner in which these products are used), and scrubbing products or exfoliants for either the face or the hands (when the composition contains exfoliant particles).

The use as a scrubbing or exfoliant product generally entails applying the product to the face, the hands or the body, rubbing for one or two minutes and then rinsing. The skin is then smooth, soft and scrubbed.

The uses indicated above can be performed in the usual manner in which products with similar effects are used.

A subject of the present invention is also the use of the composition as defined above, for removing makeup from and/or for cleansing the skin, the hair and/or mucous membranes, or for caring for the skin.

Preferably, the composition in accordance with the invention is white in the bulk, i.e. it is white in the container containing it, provided that it does not contain, in addition to interference particles, standard dyes or coloring pigments that might color the bulk. The addition of such dyes or coloring pigments is possible to modify the color of the composition, but this addition preferably does not interfere with the color-change effect on application to the skin. Preferably, the composition according to the invention is free of pigments with a screening effect, and in general is free of mineral and organic UV-screening agents.

When it contains neither dye nor coloring pigment, the appearance of the composition as seen in the bulk is preferably colorless (white) or slightly nacreous. Thus, the composition according to the invention is also characterized by lightness L* and saturation C* colorimetric values measured in the CIE 1976 colorimetric space, these values being measured in the absence of dye and/or of coloring pigment in the composition.

The calorimetric measurements L* and C* may be performed using a Minolta CR400® colorimetric chromameter. As indicated above, these calorimetric measurements should be performed on the composition containing the interference particles, but before addition of any dye or coloring pigment.

To perform the measurements, the test composition is introduced into a 15 ml jar (aperture diameter: 1.9 cm; depth: 1.8 cm). The surface of the composition introduced is smoothed off by levelling with a glass slide. The colorimeter cell is then placed in contact with this surface and the calorimetric parameters are determined. In the absence of dye and/or of coloring pigment, the compositions according to the invention are characterized by a lightness L* of greater than 60, preferably greater than 75 and more preferentially greater than 80, combined with a saturation C* of less than 10, preferably less than 5 and more preferentially less than 3, which shows that the composition is white.

For information, the white reference supplied with the Chromameter CR400® calorimeter is characterized by the following lightness L* and saturation C* values: L*=96.94±0.01, C*=2.83±0.01.

The compositions of the invention are preferably characterized by color changes between the color in the jar, the color on application to the skin, and the color after massaging on the skin. These color changes depend on the direction of the emulsion, the shear of the composition on the skin, the thickness of the coat deposited on the skin, the physical state of the product on the skin, for example if the product is in two phases after deposition on the skin (water on one side, oil on the other), or if the composition has remained in emulsion form on the skin.

In one use of the composition of the invention, it is placed on the skin as a thick coat, for example a coat of from 15 to 35 mg/cm², and, at the time of massaging on the skin, it is spread out to have a thin coat. The phenomena produced are different depending on whether the composition is in the form of an oil-in-water (O/W) emulsion (direct emulsion with oily phase dispersed in the aqueous phase) or in the form of a water-in-oil (W/O) emulsion (inverse emulsion with aqueous phase dispersed in the oily phase).

During massaging, the O/W emulsions break on the skin, releasing the oil, whereas the W/O emulsions break releasing water. Thus, there are two possible outcomes depending on the direction of the emulsion: in the case of O/W emulsions, there is a color when the product is in a thick coat, followed by disappearance of the color. The user knows that the thickness of the coat applied and thus the amount of composition applied to the skin are sufficient if a color is visible to the eye. The thick coat is applied to the entire area to be treated, for example to be freed of makeup, for example to the entire face. After application, the product is massaged and the color disappears at the same time that the oil is released, and thus, when the color is no longer perceptible by the user, this means that the massaging can be stopped and that the released oil will allow the makeup removal or the care effect.

In the case of a W/O emulsion, there is no color during the application to the skin as a thick coat, but the color appears when the emulsion breaks releasing the water and the interference particles onto the surface of the skin, which means that the massaging can be stopped.

Next, in both cases, rinsing is performed such that no shiny particles remain on the skin, the interference particles having acted as makeup-removal, cleansing or care indicator, but not remaining on the skin after rinsing.

A subject of the invention is also a process for removing makeup from and/or for cleansing the skin and/or the hair and/or mucous membranes, or for caring for the skin, comprising:

1) applying, to the area to be freed of makeup, cleansed or cared for, the composition according to the invention in a sufficient thickness, for example a coat from 15 to 35 mg/cm² thick,

2) massaging the composition into the area to spread it out until the color has disappeared if the composition is an O/W emulsion, or until the color has appeared if the composition is a W/O emulsion,

3) rinsing until the composition has been removed.

Interference Particles

For the purposes of the present invention, the term “interference particle” denotes any particle generally having a multilayer structure such that it allows the creation of a color effect by interference of light rays, which diffract and diffuse differently depending on the nature of the coats. Thus, these particles may have colors that vary according to the angle of observation and the incidence of the light.

For the purposes of the present invention, the term “multilayer structure” is intended to denote either a structure formed from a substrate covered with a single coat or a structure formed from a substrate covered with at least two or even several consecutive coats.

The interference particles used according to the invention include nacres, i.e. particles of mica. These micas are coated with metal oxide. The color of the particle depends on the thickness of the metal oxide coat. Preferably, the nacres are micas coated with titanium oxide (TiO₂) as metal oxide.

| Color of the pigment | Thickness of TiO₂ |
|---|---|
| Silver | 40 to 60 nm |
| Gold | 60 to 80 nm |
| Red | 80 to 100 nm |
| Blue | 100 to 140 nm |
| Green | 120 to 160 nm |

The thickness of the metal oxide coat and especially of the titanium oxide coat in the nacres used according to the invention is preferably greater than 80 nm, which allows the formation of a color other than silver or gold. The interference particles may also comprise a coat of another metal oxide such as tin oxide and/or silica (silicon oxide, SiO₂), in order to modify the color obtained by the mica coated with a coat of titanium oxide. Thus, for example, the color may be modified as a function of the thickness of the tin oxide coat; a coat of silica (SiO₂) may also be added to increase the amount of reflected light.

Moreover, the maximum length of the nacre used according to the invention preferably does not exceed 130 μm, in order to obtain a uniform color on spreading (the particles are visible if the size is greater). The term “maximum length” means the longest length of the particles, it being understood that the commercially available particles are a mixture of particles having different lengths, as is seen in the list of raw materials indicated later. In the composition according to the invention, the maximum particle length should not exceed 130 μm. Comparative Example 6 given below shows that, when the maximum length exceeds 130 μm, the composition obtained does not give the desired effect. Preferably, the length of the nacre ranges from 5 to 130 μm and better still from 5 to 125 μm.

As interference particles that are particularly suitable for the composition of the invention, mention may be made of nacres of the type such as:

mica/titanium oxide/tin oxide, for instance those sold under the names Timiron Silk Blue®, Timiron Silk Red®, Timiron Silk Green® and Timiron Silk Violet®, by the company Merck, the product sold under the name Xirona® Volcanic Fire by the company Merck, or those of the range Total Prestige®, Silk Orange, Silk Red, Silk Violet, Silk Blue and Silk Green sold by the company Eckart;

mica/titanium oxide, for instance those sold under the names Flamenco Blue®, Flamenco Red® and Flamenco Green® by the company Engelhard,

and mixtures thereof.

These interference particles may be present in the composition of the invention in any effective amount, such as an amount ranging, for example, from 0.5% to 5% by weight and preferably from 1% to 4% by weight relative to the total weight of the composition.

Emulsion

The emulsion constituting the support for the composition comprises an aqueous phase and an oily phase and, depending on the direction of the emulsion, the composition may be in the form of an O/W emulsion or a W/O emulsion.

The emulsion may be prepared via any emulsification process. In the case of O/W emulsions, it may also be obtained via the phase inversion temperature technique.

The emulsions according to the invention have a greater amount of inner phase than of outer phase. Thus, they contain an amount of inner phase of greater than 50% by weight relative to the total weight of the composition (oily phase for the O/W emulsions and aqueous phase for the W/O emulsions), preferably more than 60%, more preferentially from 60% to 95% and more preferentially from 60% to 90% by weight of dispersed inner phase relative to the total weight of the composition. The emulsion thus obtained breaks easily on the surface of the skin during massaging. This change on the skin induces color variations that are more visible than in the case of emulsions that do not break on the surface of the skin.

The oily phase contains at least one oil, especially a cosmetic oil. The term “oil” means a fatty substance that is liquid at room temperature (25° C.). As indicated above, the relative amount of the two phases depends on the direction of the emulsion. For the O/W emulsions, the amount of oily phase may range, for example, from more than 50% to 95% by weight, preferably from 55% to 95% by weight and better still from 60% to 90% by weight relative to the total weight of the composition. For the W/O emulsions, the amount of oily phase may range, for example, from 5% to less than 50%, preferably from 5% to 45% by weight and better still from 10% to 40% by weight relative to the total weight of the composition.

For the aqueous phases, the amounts are inverted, i.e. for the O/W emulsions, the amount of aqueous phase may range, for example, from 5% to less than 50%, preferably from 5% to 45% by weight and better still from 10% to 40% by weight relative to the total weight of the composition. For the W/O emulsions, the amount of aqueous phase may range, for example, from more than 50% to 95% by weight, preferably from 55% to 95% by weight and better still from 60% to 90% by weight relative to the total weight of the composition.

Oily Phase

The oily phase of the composition according to the invention contains all the fatty substances and lipophilic additives. It contains at least one oil, especially a cosmetic oil. The term “oil” means a fatty substance that is liquid at room temperature (25° C.). According to one preferred embodiment of the invention, the oily phase contains at least one oil chosen from hydrocarbon-based oils, fatty acid esters (or fatty esters) and fatty alkyl ethers (or fatty ethers), and mixtures thereof. The term “hydrocarbon-based oil” means any oil mainly comprising carbon and hydrogen atoms, and possibly ester, ether, fluoro, carboxylic acid and/or alcohol groups:

the linear or branched hydrocarbon-based oils, of mineral, synthetic or animal origin, are chosen from liquid paraffins (mineral oil) and derivatives thereof, petroleum jelly, liquid petroleum jelly, perhydrosqualene, polydecenes, isohexadecane, isododecane, and hydrogenated polyisobutene (or hydrogenated isoparaffin) such as Parleam oil;

the fatty esters are preferably those obtained from a linear- or branched-chain alcohol, containing from 1 to 17 carbon atoms, and from a linear- or branched-chain fatty acid, containing from 3 to 18 and preferably from 12 to 17 carbon atoms. Advantageously, the ester is a saturated ester not containing any ether or hydroxyl groups (the total amount of carbon in the ester may range from 12 to 50 and preferably from 20 to 50).

Examples of fatty esters that may be mentioned include 2-ethylhexyl caprate/caprylate (or octyl caprate/caprylate), ethyl laurate, butyl laurate, hexyl laurate, isohexyl laurate, isopropyl laurate, methyl myristate, ethyl myristate, butyl myristate, isobutyl myristate, isopropyl myristate, 2-octyldodecyl myristate, 2-ethylhexyl monococoate (or octyl monococoate), methyl palmitate, ethyl palmitate, isopropyl palmitate, isobutyl palmitate, 2-ethylhexyl palmitate (or octyl palmitate), butyl stearate, isopropyl stearate, isobutyl stearate, isocetyl stearate, isotearyl isostearate, isopropyl isostearate, 2-ethylhexyl stearate (or octyl stearate), 2-ethylhexyl pelargonate (or octyl pelargonate), 2-ethylhexyl hydroxystearate (or octyl hydroxystearate), decyl oleate, diisopropyl adipate, bis(2-ethylhexyl)adipate (or dioctyl adipate), diisocetyl adipate, 2-ethylhexyl succinate (or octyl succinate), diisopropyl sebacate, 2-ethylhexyl malate (or octyl malate), pentaerythrityl caprate/caprylate, pentaerythritol tetraisostearate, 2-ethylhexyl hexanoate (or octyl hexanoate), octyldodecyl octanoate, isodecyl neopentanoate, isostearyl neopentanoate, cetearyl isononanoate,. isodecyl isononanoate, isononyl isononanoate, isotridecyl isononanoate, lauryl lactate, myristyl lactate, cetyl lactate, myristyl propionate, 2-ethylhexyl 2-ethylhexanoate (or octyl 2-ethylhexanoate), 2-ethylhexyl octanoate (or octyl octanoate) and mixtures thereof, C12-C15 fatty alkyl benzoates (Finsolv TN from Finetex), isopropyl lauroyl sarcosinate (Eldew SL 205 from Unipex) and dicaprylyl carbonate (Cetiol CC from the company Cognis);

a fatty ether that may be mentioned is dicaprylyl ether (Cetiol OE from the company Cognis).

The composition may also contain one or more other oils chosen from plant oils, silicone oils and fluoro oils, which may be volatile or non-volatile. Mention may be made especially of:

hydrocarbon-based oils of plant origin such as sweet almond oil, avocado oil, castor oil, coriander oil, olive oil, jojoba oil, sesame oil, groundnut oil, grapeseed oil, rapeseed oil, coconut oil, hazelnut oil, shea butter, palm oil, apricot kernel oil, beauty-leaf oil, rice bran oil, corn germ oil, wheatgerm oil, soybean oil, sunflower oil, evening primrose oil, safflower oil, passionflower oil, rye oil, and caprylic/capric acid triglycerides, for instance those sold by the company Stearineries Dubois or those sold under the names Miglyol 810, 812 and 818 by the company Dynamit Nobel;

volatile or non-volatile silicone oils, for instance volatile or non-volatile polydimethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates and polymethylphenylsiloxanes;

fluoro oils, such as partially hydrocarbon-based and/or silicone-based fluoro oils, for instance those described in document JP-A-2 295 912.

The oily phase of the composition according to the invention may also comprise fatty substances other than oils, and especially one or more compounds chosen from waxes, gums and pasty fatty substances, these compounds possibly being of plant, animal, mineral or synthetic origin, and silicone or non-silicone based. The waxes may be hydrocarbon-based waxes, silicone waxes and/or fluoro waxes, optionally comprising ester or hydroxyl functions. The wax(es) may represent, for example, from 0 to 10% by weight and especially from 0.05% to 5% by weight relative to the total weight of the composition.

Aqueous Phase

The aqueous phase of the compositions according to the invention may contain, besides water, one or more solvents chosen from monoalcohols containing from 1 to 6 carbon atoms, and polyols, and mixtures thereof. A monoalcohol that may especially be mentioned is ethanol. Polyols that may especially be mentioned include glycerol; glycols, for instance butylene glycol, isoprene glycol, propylene glycol and polyethylene glycols such as PEG-8; sorbitol; sugars such as glucose, fructose, maltose, lactose and sucrose; and mixtures thereof. When they are present, the amount of monoalcohols and of polyols in the composition of the invention may range, for example, from 0.01% to 30% by weight, preferably from 2% to 25% by weight and better still from 5% to 20% by weight relative to the total weight of the composition.

Emulsifiers

The composition according to the invention constitutes an emulsion, and the dispersion of one phase in the other is preferably performed in the presence of at least one emulsifier that is different depending on the direction of the emulsion. The amount of emulsifier may range, for example, from 1% to 20% by weight, preferably from 1% to 15% by weight and better still from 2% to 10% by weight relative to the total weight of the composition.

Depending on the direction of the emulsion, the emulsifier may be introduced into the aqueous phase or into the oily phase.

The O/W emulsions preferably comprise an emulsifier or a mixture of emulsifiers with an HLB (hydrophilic-lipophilic balance) ranging from 8 to 18. Moreover, the solubility of certain emulsifiers in the oils may optionally increase with the temperature, which allows emulsions to be obtained via the phase inversion temperature process.

The W/O emulsions preferably comprise an emulsifier or a mixture of emulsifiers with an HLB (hydrophilic-lipophilic balance) of less than 8.

The emulsifiers of the O/W emulsions may be chosen from ethoxylated fatty alcohols, ethoxylated fatty acids, fatty acid esters of a sugar or of sorbitan, ethoxylated fatty acid partial glycerides, ethoxylated or non-ethoxylated polyglycerolated fatty acid triglycerides, and mixtures thereof, and any other suitable standard emulsifier.

The preferred emulsifiers are ethoxylated fatty alcohols or ethoxylated fatty acids having the following formulae:
R—O—(CH₂—CH₂—O)ₘH
or
R—COO—(CH₂—CH₂—O)ₘH
in which R is a linear or branched, saturated or unsaturated hydrocarbon-based chain with a number of carbons ranging from 10 to 24 and m is between 8 and 100.

The ethoxylated alcohols are fatty alkyl ethers comprising, for example, from 10 to 100 and preferably from 10 to 50 oxyethylene groups, such as Laureth-10 to 12, Ceteth-10 to 30, Steareth-10 to 30, Ceteareth-10 to 30, Isosteareth-10 to 50 and Beheneth-10 to 50.

The ethoxylated acids are fatty acid esters comprising, for example, from 10 to 150 and preferably from 10 to 100 oxyethylene groups, such as PEG-10 to PEG-50 laurate, palmitate, stearate, palmitostearate or behenate. Mention may be made more particularly of polyethylene glycol stearates, for instance PEG-100 stearate, PEG-50 stearate and PEG-40 stearate; and mixtures containing them, such as the mixture of glyceryl stearate and of PEG-100 stearate, sold under the name Arlacel 165 by the company Uniqema and under the name Simulsol 165 by the company SEPPIC; oxyethylenated fatty acid esters of sorbitan comprising, for example, from 20 to 100 OE, for example those sold under the trade names Tween 20 or Tween 60 by the company Uniqema; oxyethylenated sugar esters, for instance PEG-20 methylglucose sesquistearate.

Fatty acid esters of a sugar or of sorbitan that may be mentioned include sucrose esters such as sucrose stearate, and sorbitan esters such as the sorbitan palmitate sold under the name Span 40 by the company Uniqema.

Emulsifiers that may also be mentioned include dimyristyl tartrate and mixtures containing it, such as the mixture of dimyristyl tartrate, cetearyl alcohol, Pareth-7 and PEG-25 laureth-25, sold under the name Cosmacol PSE by the company Sasol (CTFA name: Dimyristyl tartrate/cetearyl alcohol/12-15 Pareth-7/PPG-25 laureth-25)

Other emulsifiers may be chosen from fatty acid mono- or diglycerides, polyglycerolated fatty acids or ethoxylated triglycerides and other emulsifiers conventionally used such as alkylpolyglucosides, especially those sold under the name Montanov by the company SEPPIC.

Co-emulsifiers may be added to these emulsifiers, for instance fatty alcohols containing from 12 to 30 carbon atoms, for instance cetyl alcohol, stearyl alcohol and a mixture thereof (cetearyl alcohol), and behenyl alcohol, and mixtures thereof.

As emulsifiers that may be used for the W/O emulsions, examples that may be mentioned include fatty acid esters of polyethylene glycol of suitable HLB, such as PEG-30 dipolyhydroxystearate sold under the name Arlacel P135 by the company Uniqema; glycerolated derivatives, for instance polyglyceryl-4 isostearate; polyisobutenyl succinate derivatives, for instance the mixture TEA-diethanolaminoethyl polyisobutenyl succinate/ethylhexyl palmitate, sold under the name Chemcinnate 2000 by the company Chemron; oligomers or polymers derived from polyolefin, for instance the products sold under the names Lubrizol 2724 and Lubrizol 5603 by the company Lubrizol; silicone surfactants, for instance dimethicone copolyols such as the mixture of cyclomethicone and of dimethicone copolyol, sold under the names DC 5225 C and DC 3225 C by the company Dow Corning, and such as alkyldimethicone copolyols, such as the laurylmethicone copolyol sold under the name “Dow Corning 5200 Formulation Aid” by the company Dow Corning, cetyldimethicone copolyol sold under the name Abil EM 90® by the company Goldschmidt and the mixture of polyglyceryl-4 isostearate/cetyldimethicone copolyol/-hexyllaurate sold under the name Abil WE 09® by the company Goldschmidt. One or more co-emulsifiers may also be added thereto, which may advantageously be chosen from the group comprising esters of a branched-chain fatty acid and of a polyol, and especially esters of a branched-chain fatty acid and of glycerol and/or sorbitan, for example polyglyceryl isostearate, such as the product sold under the name Isolan GI 34 by the company Goldschmidt, sorbitan isostearate, such as the product sold under the name Arlacel 987 by the company Uniqema, sorbitan glyceryl isostearate, such as the product sold under the name Arlacel 986 by the company Uniqema, and mixtures thereof.

Additives

The composition according to the invention may contain various water-soluble and/or liposoluble additives or active agents, including those chosen from those conventionally used in products for caring for or for removing makeup from the skin, in so far as these additives and the amounts thereof do not harm the desired qualities of the composition according to the invention.

Adjuvants that may be mentioned in particular include mineral or organic fillers, after it has been confirmed that they do not harm the color variation of the products.

The mineral and organic, porous or non-porous, spherical or non-spherical fillers may be chosen from mineral oxides or organic fillers.

Among the mineral oxides that may be mentioned are titanium oxide, zinc oxide, iron oxide, aluminium oxide, zirconium oxide, selenium oxide, strontium oxide, manganese oxide and, more preferentially, oxides of silicon including silicas. The silica may be chosen from hydrophilic silicas and hydrophobic silicas, and mixtures thereof. The term “silica” means both pure silicas (hydrophilic or hydrophobic) and silica-coated particles. These silicas are preferably amorphous and they may be of pyrogenic origin or of precipitated origin. They may be in pulverulent form or in the form of an aqueous dispersion. They are preferably characterized by a specific surface area of from 30 to 500 m²/g, a mean number-average particle size ranging from 3 to 50 nm and a packed density ranging from 40 to 200 g/l and better still from 50 to 150 g/l. Examples of silicas that may be mentioned include those sold under the names Aerosil 90®, 130®, 150®, 200®, 300®, 380®, OX 50®, FK 320 DS®, R202®, R805®, R812®, R972® and R974® by the company Degussa-Hüls.

Other mineral fillers may be used. These are, for example, aluminium silicates (montmorillonites or kaolinites), magnesium silicates (talc), aluminium magnesium silicates (bentonites, clays, attapulgites and smectites), calcium magnesium silicates (dolomites), and aluminium sodium silicates. Thus, it is possible to select, for example, the talc sold under the name Luzenac 15 M00® by the company Luzenac, or kaolin or aluminium silicate sold under the name Kaolin Supreme® by the company Imerys.

The organic fillers may be chosen, for example, from:

synthetic polymer microspheres, for instance those of Nylon (Orgasol 2002 UD NAT COS® from the company Atochem), those based on vinylidene chloride/acrylonitrile/methacrylonitrile copolymer containing isobutane, and expanded (Expancel 551 DE® from the company Expancel);

Nylon 6,6 fibres (Polyamide 0.9 dtex 0.3 mm from Etablissements Paul Bonte), cellulose or “Rayon” fibres (Rayon Flock RCISE NOOO3 MO4® from the company Claremont) or starch fibres (Amidon de Maïs B® from the company Roquette).

Examples of additives other than fillers that may be mentioned include the adjuvants that are common in cosmetics, such as foaming or detergent surfactants, which are either anionic, for instance sodium lauryl ether sulfates, sodium alkyl phosphates or sodium trideceth sulfate, or amphoteric, for instance alkyl betaines or disodium cocoamphodiacetate, or nonionic with an HLB of greater than 10, for instance POE/PPG/POE (CTFA name: Poloxamer), alkyl polyglucosides (or APG) and polyglyceryl-3 hydroxylauryl ether; preserving agents; sequestering agents (EDTA and salts thereof); antioxidants; fragrances; dyestuffs, soluble dyes or encapsulated or non-encapsulated pigments; sunscreens; anionic, nonionic, cationic or amphoteric, hydrophilic or lipophilic polymers, thickeners or dispersants; hydrophilic or lipophilic cosmetic or dermatological active agents. The amounts of these various adjuvants are those conventionally used in the field under consideration, for example from 0.01% to 20% of the total weight of the composition. These adjuvants and the amounts thereof must be such that they do not modify the desired property for the composition of the invention.

As active agents that are common in cosmetics or dermatology, which may be used according to the invention, examples that may be mentioned include vitamins, for instance vitamins A, B3, PP, B5, E, K1 and/or C and derivatives of these vitamins and especially esters thereof; keratolytic or pro-desquamating agents, for example a-hydroxy acids, β-hydroxy acids, α-keto acids and β-keto acids, retinoids and esters thereof, retinal and retinoic acid and its derivatives; plant extracts such as extracts of ruscus and/or of common horsechestnut; xanthine bases such as caffeine; free-radical scavengers; sunscreens; moisturizers, for instance polyols; ceramides; DHEA and its derivatives; coenzyme Q10; agents for bleaching and depigmenting via a biological action, for instance kojic acid, extracts of scullcap, mulberry, liquorice and/or camomile; para-aminophenol derivatives, arbutin and derivatives thereof, and mixtures thereof.

For use in the cosmetic treatment of greasy or combination skin, the composition according to the invention will in particular contain at least one active agent chosen from vitamins B3 and B5; zinc salts, and in particular zinc oxide and zinc gluconate; salicylic acid and its derivatives such as 5-n-octanoylsalicylic acid; triclosan; capryloylglycine; an extract of clove; octopirox; hexamidine; and azeleic acid and its derivatives. In the event of incompatibility or to stabilize them, the active agents mentioned above may be incorporated into spherules, especially ionic or nonionic vesicles and/or nanoparticles (nanocapsules and/or nanospheres).

The emulsions according to the invention may be prepared via any standard emulsification process.

According to one particular embodiment of the invention, when they are O/W emulsions, they may be obtained via the phase inversion process. This preparation process comprises:

1) weighing out all the constituents of the composition (except for the heat-sensitive raw materials and the fillers) in a container;

2) homogenizing the mixture, for example using a Rayneri blender at 350 rpm, and heating by gradually raising the temperature using a water bath, up to a temperature greater than or equal to the phase inversion temperature T2, i.e. until a transparent or translucent phase is obtained (microemulsion or lamellar phase zone), followed by a more viscous white phase, which indicates that the inverse emulsion (W/O) has been obtained;

3) stopping the heating and continuing the stirring until the mixture has cooled to room temperature, passing through the phase inversion temperature T1, i.e. the temperature at which a fine O/W emulsion is formed;

4) when the temperature has fallen below the phase inversion temperature zone (T1), adding the fillers and, if any, the heat-sensitive raw materials.

A stable O/W emulsion whose oil droplets are very fine (volume-average size of the oil globules ranging from 50 nm to 1000 nm, preferably from 70 nm to 350 nm and more particularly from 70 to 300 nm) is obtained.

The examples indicated below will allow the invention to be understood more clearly without, however, being limiting in nature. The amounts indicated are weight percentages, unless otherwise mentioned. The names are given as the chemical name, the commercial name and the CTFA name, depending on the compound.

### EXAMPLES 1 TO 9

Examples 1 to 5 are examples according to the invention, while Examples 6 to 9 are comparative examples. All these examples are O/W emulsions. These examples were prepared according to the phase inversion process described above.

In each example, a different nacre was introduced in an amount of 1.5%. The compositions obtained were spread in an amount of 15 mg/cm² onto a black and white contrast card in order to determine the color effect thereof that will be obtained on the skin. The results are given in Tables 1 and 2 presented below:

The characteristics of the nacres used in these examples are as follows:

| | | | Nacre | |
|---|---|---|---|---|
| | | | size = | |
| | | | minimum | |
| | | | length/ | |
| | Composition and | | maximum | Thickness |
| Ex. No. | commercial name | Supplier | length | of TiO₂ |
| 1 | Mica/titanium | Merck/Rona | 5-25 μm | 80-100 nm |
| | dioxide/tin | | | |
| | oxide (58/41/1) | | | |
| | (Timiron Silk | | | |
| | Red) | | | |
| 2 | Mica/titanium | Merck/Rona | 5-25 μm | 100-140 nm |
| | dioxide/tin | | | |
| | oxide (35/64/1) | | | |
| | (Timiron Silk | | | |
| | Blue) | | | |
| 3 | Mica/titanium | Merck/Rona | 10-60 μm | 80-100 nm |
| | dioxide/silica/ | | | |
| | tin oxide | | | |
| | (24/40.5/35/0.5) | | | |
| | (Xirona Volcanic | | | |
| | Fire) | | | |
| 4 | Mica/titanium | Taizhu | 10-60 μm | 80-100 nm |
| | dioxide/tin | | | |
| | oxide (KTZ | | | |
| | Interval Red) | | | |
| 5 | Mica/titanium | Merck/Rona | 10-125 μm | 80-100 nm |
| | dioxide/tin | | | |
| | oxide (70/29/1) | | | |
| | (Timiron | | | |
| | Starlight Red) | | | |
| 6 | Mica/titanium | Eckart | 20-150 μm | 80-100 nm |
| | dioxide/tin | | | |
| | oxide | | | |
| | (63.5/35/5) | | | |
| | (Prestige | | | |
| | Sparkling Red) | | | |
| 7 | Mica/titanium | Merck/Rona | 5-25 μm | 40-60 nm |
| | dioxide (62/38) | | | |
| | (Timiron Super | | | |
| | Sheen 1001) | | | |
| 8 | Mica/titanium | Merck/Rona | 10-60 μm | 80-100 nm |
| | dioxide/carmine | | | |
| | (58/40/2) | | | |
| | (Colorona | | | |
| | Carmine Red) | | | |
| 9 | Mica/black iron | Merck/Rona | 10-60 μm | none |
| | oxide (48/52) | | | |
| | (Colorona | | | |
| | Blackstar Red) | | | |

TABLE 1
Examples 1 to 5 according to the invention

| | | | | | |
|---|---|---|---|---|---|
| Composition | Ex. 1 | Ex. 2 | Ex. 3 | Ex. 4 | Ex. 5 |

Phase A

| | | | | | |
|---|---|---|---|---|---|
| Eumulgin BA 10 ® | 5 | 5 | 5 | 5 | 5 |
| (1) | | | | | |
| Ceraphyl 368 ® (2) | 66.2 | 66.2 | 66.2 | 66.2 | 66.2 |
| Propyl paraben | 0.15 | 0.15 | 0.15 | 0.15 | 0.15 |
| Methyl paraben | 0.15 | 0.15 | 0.15 | 0.15 | 0.15 |
| Glycerol | 10 | 10 | 10 | 10 | 10 |
| Deionized water | 13.75 | 13.75 | 13.75 | 13.75 | 13.75 |

Phase B

| | | | | | |
|---|---|---|---|---|---|
| Aerosil 200 ® (3) | 1 | 1 | 1 | 1 | 1 |
| Ethanol | 2 | 2 | 2 | 2 | 2 |
| Chlorohexidine | 0.25 | 0.25 | 0.25 | 0.25 | 0.25 |
| digluconate | | | | | |
| Mica/titanium | 1.5 | | | | |
| dioxide/tin | | | | | |
| oxide (58/41/1) | | | | | |
| (Timiron Silk | | | | | |
| Red) | | | | | |
| Mica/titanium | | 1.5 | | | |
| dioxide/tin | | | | | |
| oxide (35/64/1) | | | | | |
| (Timiron Silk | | | | | |
| Blue) | | | | | |
| Mica/titanium | | | 1.5 | | |
| dioxide/silica/ | | | | | |
| tin oxide | | | | | |
| (24/40.5/35/0.5) | | | | | |
| (Xirona Volcanic | | | | | |
| Fire) | | | | | |
| Mica/titanium | | | | 1.5 | |
| dioxide/tin | | | | | |
| oxide (KTZ | | | | | |
| Interval Red) | | | | | |
| Mica/titanium | | | | | 1.5 |
| dioxide/tin | | | | | |
| oxide (70/29/1) | | | | | |
| (Timiron | | | | | |
| Starlight Red) | | | | | |
| Total | 100 | 100 | 100 | 100 | 100 |
| Viscosity (Pa · s) | 2 | 2 | 2 | 2 | 2 |
| pH | 5.9 | 5.9 | 5.9 | 5.9 | 5.9 |
| Appearance in | White | White | White | White | White nacreous |
| the jar | nacreous | nacreous | nacreous | nacreous | |
| Appearance | Pink | Blue | Pink | Pink | Pink color |
| during | color | color | color | color | |
| application to | | | | | |
| the contrast | | | | | |
| card | | | | | |

(1) Oxyethylenated (10 OE) behenyl alcohol = Eumulgin BA 10 ® sold by the company Cognis
(2) Ethylhexyl palmitate = Ceraphyl 368 ® sold by the company ISP
(3) Silica = Aerosil 200 ® sold by the company Degussa-Hüls

TABLE 2
Comparative Examples 6 to 9

| | Ex. 6 | Ex. 7 | Ex. 8 | Ex. 9 |
|---|---|---|---|---|
| Composition | (comparative) | (comparative) | (comparative) | (comparative) |
| Eumulgin BA 10 ® (1) | 5 | 5 | 5 | 5 |
| Ceraphyl 368 ® (2) | 66.2 | 66.2 | 66.2 | 66.2 |
| Propyl paraben | 0.15 | 0.15 | 0.15 | 0.15 |
| Methyl paraben | 0.15 | 0.15 | 0.15 | 0.15 |
| Glycerol | 10 | 10 | 10 | 10 |
| Deionized water | 13.75 | 13.75 | 13.75 | 13.75 |
| Aerosil 200 ® (3) | 1 | 1 | 1 | 1 |
| Ethanol | 2 | 2 | 2 | 2 |
| Chlorohexidine | 0.25 | 0.25 | 0.25 | 0.25 |
| digluconate | | | | |
| Mica/titanium | 1.5 | | | |
| dioxide/tin | | | | |
| oxide | | | | |
| (63.5/35/5) | | | | |
| (Prestige | | | | |
| Sparkling Red) | | | | |
| Mica/titanium | | 1.5 | | |
| dioxide (62/38) | | | | |
| (Timiron Super | | | | |
| Sheen 1001) | | | | |
| Mica/titanium | | | 1.5 | |
| dioxide/carmine | | | | |
| (58/40/2) | | | | |
| (Colorona | | | | |
| Carmine Red) | | | | |
| Mica/black iron | | | | 1.5 |
| oxide (48/52) | | | | |
| (Colorona | | | | |
| Blackstar Red) | | | | |
| Total | 100 | 100 | 100 | 100 |
| Viscosity | 2 | 2 | 2 | 2 |
| (Pa · s) | | | | |
| pH | 5.9 | 5.9 | 5.9 | 5.9 |
| Appearance in | White | White | Pink | Dark |
| the jar | nacreous | nacreous | | brown |
| Appearance | Weak pink | No | No | No color |
| during | effect | color | color | change |
| application to | insufficient | effect | change | |
| the contrast | | | | |
| card | | | | |

(1) Oxyethylenated (10 OE) behenyl alcohol = Eumulgin BA 10 ® sold by the company Cognis
(2) Ethylhexyl palmitate = Ceraphyl 368 ® sold by the company ISP
(3) Silica = Aerosil 200 ® sold by the company Degussa-Hüls

Examples 1 to 5 show that the color effect is obtained for a TiO₂ thickness of greater than 80 nm (silver and gold colors excluded), whereas with a thickness of less than 80 nm (Comparative Example 7), the desired result cannot be achieved.

Examples 1 to 5 show that the color-change effect cannot be obtained unless the nacre has a maximum length of less than 130 μm, whereas with a longer length (Comparative Example 6), the effect cannot be obtained.

Comparative Examples 8 and 9 show that the color effect cannot be obtained if the nacres are colored, and that it is therefore preferable to use uncolored nacres.

| | |
|---|---|
| Composition | Ex. 10 |

Phase A

| | |
|---|---|
| Ceraphyl 368 ® (2) | 45.5 |
| Dow Corning 245 Fluid ® (4) | 14 |
| Marcol 82 ® (5) | 10 |
| Parleam ® (6) | 9 |

Phase B

| | |
|---|---|
| Deionized water | qs 100 |
| Glucamate SSE 20 (7) | 4.6 |
| Glycerol | 11 |
| Preserving agent | qs |
| Mica/titanium dioxide/tin | 1.5 |
| oxide (58/41/1) (Timiron | |
| Silk Red) | |
| Viscosity (Pa · s) | 2 |
| pH | 5.9 |
| Appearance | Nacreous soft white gel- |
| | cream with green tints |
| Appearance during | Green color |
| application to the skin | |

(2) Ethylhexyl palmitate = Ceraphyl 368 ® sold by the company ISP
(4) Cyclopentasiloxane = Dow Corning 245 Fluid ® sold by the company Dow Corning
(5) Mineral oil = Marcol 82 ® sold by the company Esso
(6) Hydrogenated polyisobutene = Parleam ® sold by the company NOF Corporation
(7) PEG-20 methylglucose sesquistearate = Glucamate SSE 20 ® sold by the company Chemron

The procedure is as follows:

1—the constituents of phase A are mixed together,

2—the constituents of phase B are mixed together,

3—phases A and B are heated to 80° C.,

4—the aqueous phase B is gently introduced with rapid stirring into the oily phase A.

An O/W emulsion is obtained.

### EXAMPLE 11

### According to the Invention: Makeup-Removing Cream (W/O Emulsion)

| | |
|---|---|
| Composition | Ex. 11 |

Phase A

| | |
|---|---|
| Marcol 82 ® (5) | 3.2 |
| Isopropyl Palmitate ® (8) | 3 |
| Dow Corning 5225C | 9.2 |
| Formulation Aid ® (9) | |

Phase B

| | |
|---|---|
| Deionized water | qs 100 |
| PEG-8 | 4 |
| Ethanol | 2.5 |
| Glycerol | 5 |
| Preserving agent | 0.65 |
| Na salt of EDTA | 0.1 |
| NaCl | 2.5 |
| Timiron Starlight Red ® | 3.5 |
| Appearance | White product |
| Appearance during | White product then |
| application to the skin | appearance of pink “pearls” |
| | during the release of water |
| | in the form of droplets |

(5) Mineral oil = Marcol 82 ® sold by the company Esso
(8) Isopropyl palmitate = Isopropyl Palmitate ® sold by the company Cognis
(9) Cyclopentasiloxane (and) PEG/PPG-18/18 dimethicone = Dow Corning 5225C Formulation Aid ® sold by the company Dow Corning

Procedure: Phase A is prepared by mixing the constituents together with stirring at 600 rpm. Phase B is prepared separately and a portion (about 1/10) of phase B is introduced into phase A very slowly with stirring. The rest of phase B is then added more quickly, with continued stirring, and stirring is continued for a certain time.

A white cream with a viscosity measured using a Rheomat 180 viscometer, spindle 4, of 5.74 Pa·s (57.4 poises) at time zero is obtained. This viscosity stabilizes after 10 minutes to 4.56 Pa·s (45.6 poises).

### EXAMPLE 12

### According to the Invention: W/O Makeup-Removing Cream

| | |
|---|---|
| Composition | Ex. 12 |

Phase A

| | |
|---|---|
| L2724 ® (10) | 2.5 |
| Isohexadecane | 3.29 |
| Parleam ® (6) | 2.47 |
| Cyclomethicone | 1.64 |
| Preserving agent | 0.1 |

Phase B

| | |
|---|---|
| Deionized water | qs 100 |
| Magnesium sulfate | 0.9 |
| Preserving agent | 0.65 |
| Timiron Silk Blue ® | 3 |
| Appearance | White product |
| Appearance during | White product then |
| application to the skin | appearance of pink “pearls” |
| | during the release of water |
| | in the form of droplets |

(6) Hydrogenated polyisobutene: Parleam ® sold by the company NOF Corporation
(10) Polyolefin with succinic end groups: L2724 ® sold by the company Lubrizol

Procedure: the aqueous phase is introduced into the oily phase comprising the oils and the emulsifiers, with stirring, at a temperature preferably ranging from about 20 to 60° C.

### EXAMPLE 13

### According to the Invention: O/W Emulsion

| | |
|---|---|
| Composition | Ex. 13 |

Phase A

| | |
|---|---|
| Eumulgin BA 10 ® (1) | 5 |
| Ceraphyl 368 ® (2) | 66.2 |
| Propyl paraben | 0.15 |
| Methyl paraben | 0.15 |
| Glycerol | 10 |
| Deionized water | 13.75 |

Phase B

| | |
|---|---|
| Aerosil 200 ® (3) | 1 |
| Ethanol | 2 |
| Chlorohexidine digluconate | 0.25 |
| Mica/titanium dioxide/tin | 1.5 |
| oxide (58/41/1) (Timiron | |
| Silk Red) | |
| 34 PC 3516 Ultra Blue BC ® | 0.002 |
| from Noveon (dye) | |
| Total | 100 |
| Viscosity (Pa · s) | 2 |
| pH | 5.9 |
| Appearance in the jar | Nacreous blue |
| Appearance during | Violet color |
| application to the black | |
| part of the contrast card | |

(1) Oxyethylenated (10 OE) behenyl alcohol = Eumulgin BA 10 ® sold by the company Cognis
(2) Ethylhexyl palmitate = Ceraphyl 368 ® sold by the company ISP
(3) Silica = Aerosil 200 ® sold by the company Degussa-Hüls

The above written description of the invention provides a manner and process of making and using it such that any person skilled in this art is enabled to make and use the same, this enablement being provided in particular for the subject matter of the appended claims, which make up a part of the original description and including a rinse-off cosmetic composition in the form of an emulsion containing an oily phase and an aqueous phase, wherein it contains hydrophilic interference particles chosen from uncolored nacres coated with one or more coats of one or more metal oxides, these nacres having a maximum length of less than or equal to 130 μm, and the thickness of metal oxides being greater than 80 nm, the amount of inner phase being greater than 50% by weight relative to the total weight of the composition. As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A rinse-off composition in the form of an emulsion comprising inner and outer phases, one of which being an oily phase and the other of which being an aqueous phase, said composition comprising hydrophilic interference particles chosen from uncolored nacres coated with one or more coats of one or more metal oxides, said nacres having a maximum length of less than or equal to 130 μm, the total thickness of the one or more metal oxide coats being greater than 80 nm, the amount of inner phase being greater than 50% by weight relative to the total weight of the composition.

2. The composition according to claim 1, wherein, in the absence of any dye and/or coloring pigment, the composition has a lightness L* of greater than 60 and a saturation C* of less than 10, measured in the CIE 1976 calorimetric space.

3. The composition according to claim 1, wherein the nacres have a length ranging from 5 to 130 μm.

4. The composition according to claim 1, wherein the particles consist of mica coated with a coat of titanium oxide.

5. The composition according to claim 1, wherein the interference particles consist of mica coated with a coat of titanium oxide and a coat of tin oxide and/or of silicon dioxide.

6. The composition according to claim 1, wherein the interference particles are chosen from mica/titanium oxide/tin oxide nacres, mica/titanium oxide nacres, and mixtures thereof.

7. The composition according to claim 1, wherein the interference particles are present in an amount ranging from 0.5% to 5% by weight relative to the total weight of the composition.

8. The composition according to claim 1, wherein the composition is a W/O or O/W emulsion.

9. The composition according to claim 1, wherein the amount of inner phase ranges from 60% to 90% by weight relative to the total weight of the composition.

10. The composition according to claim 1, wherein the oily phase comprises at least one oil chosen from hydrocarbon-based oils, fatty acid esters and fatty alkyl ethers, and mixtures thereof.

11. The composition according to claim 1, further comprising at least one emulsifier.

12. The composition according to claim 1, wherein it is a composition for cleansing and/or for removing makeup from the skin, the hair or mucous membranes.

13. A method for removing makeup from and/or for cleansing the skin and/or the hair and/or mucous membranes, or for caring for the skin, comprising applying the composition of claim 1 to the skin and/or the hair and/or mucous membranes.

14. A process for removing makeup from and/or for cleansing the skin and/or the hair and/or mucous membranes, or for caring for the skin, which comprises:
1) applying, to the area to be freed of makeup, cleansed or cared for, the composition according to claim 1 in the form of a O/W or W/O emulsion, in a thickness of from 15 to 35 mg/cm²,
2) massaging the composition into the area to spread it out until the color has disappeared if the composition is an O/W emulsion, or until color has appeared if the composition is a W/O emulsion,
3) rinsing until the composition has been removed.
